(19) United States
(12) Patent Application Publication
(10) Pub. No.: US 2026/0148837 A1
(43) Pub. Date: May 28, 2026

(54) DEEP LEARNING-BASED MULTIPHASE CORONARY CT INTERPOLATION SYSTEM AND METHOD

(71) Applicant: INDUSTRY-ACADEMIC COOPERATION FOUNDATION, YONSEI UNIVERSITY, Seoul (KR)

(72) Inventors: Joon Sang LEE, Seoul (KR); Jung Sun KIM, Seoul (KR); Seul Gee LEE, Seoul (KR); Jun Hong KIM, Seoul (KR); Young Woo KIM, Seoul (KR); Hyeong Jun LEE, Seoul (KR)

(21) Appl. No.: 19/397,163

(22) Filed: Nov. 21, 2025

(30) Foreign Application Priority Data
Nov. 22, 2024 (KR) 10-2024-0168733
Oct. 16, 2025 (KR) 10-2025-0149607

(51) Int. Cl. G16H 30/20; G06T 3/40; G16H 30/40

(52) U.S. Cl. CPC G16H 30/20; G06T 3/40; G16H 30/40

(57) ABSTRACT

A deep learning-based multiphase coronary CT interpolation system according to an embodiment includes: an image processing unit that acquires two phases of coronary CT images as image data and performs preprocessing on the image data to generate preprocessed data; a deep learning inference unit that acquires a trained deep learning model and inputs the preprocessed data into the trained deep learning model to predict a 3D displacement map (registration field) at a specific time; and an output data generation unit that inputs the 3D displacement map to a spatial transformation layer (STL) to generate output data in the form of a CCTA image, wherein the output data includes a CCTA image for at least one arbitrary phase between the two phases of coronary CT images.

### CROSS REFERENCE TO RELATED APPLICATION

The present application claims priority to Korean Patent Application 10-2024-0168733, filed Nov. 22, 2024, and Korean Patent Application 10-2025-0149607, filed Oct. 16, 2025, in the Korean Intellectual Property Office, the entire contents of which are incorporated here for all purposes by this reference.

### BACKGROUND

The disclosure relates to a deep learning-based multiphase coronary CT interpolation system and method, and more particularly, to a deep learning-based multiphase coronary CT interpolation system and method, wherein it is possible to generate images (4D CCTA) of CCTA of intermediate phases from two different phases of coronary computed tomography angiography (CCTA) images in a cardiac cycle by utilizing deep learning.

Coronary CT Angiography (CCTA) is the standard noninvasive method for assessing coronary artery stenosis and calcification. Demand is increasing for obtaining information throughout the cardiac cycle, including the ability to quantify hemodynamics. To obtain multiphase CCTA, multiple points within the cardiac cycle are captured using electrocardiogram (ECG) gating. A representative method, 10-phase retrospective gating, allows for continuous observation of cardiac contraction and relaxation. However, 10-phase imaging increases radiation exposure by two to three times compared to single-phase imaging. This increases patient burden, test refusals, and the risk of latent cancer.

The conventional interpolation technology utilizes traditional image registration techniques such as OpticalFlow and DeformableRegistration. However, because coronary arteries are only a few millimeters in diameter, this technique leads to vessel boundary blurring and lumen volume errors. Although AI-based frame interpolation techniques have recently emerged, many studies require continuous images of 3 to 4 phases or more as learning input. As a result, this causes a vicious cycle of high-dose CT data acquisition. Attempts have been made to interpolate using only a single or two-phase approach. However, pixel regression methods fail to maintain vascular microstructure and can lead to overfitting when data is insufficient.

Furthermore, existing models fail to adequately capture nonlinear and non-periodic variations in the cardiac cycle, particularly rapid valve motion or arrhythmia. Coronary artery overlap or discontinuity in the generated intermediate phases may lead to significant errors in CT-FFR and plaque dynamics simulations.

Clinical practice demands multiphase sequences that can reliably capture hemodynamics while maintaining low doses. Technological gaps are evident in CT-FFR, dynamic perfusion analysis, and pre- and post-stent comparisons. Therefore, an interpolation technology that accurately reproduces vascular microstructure and nonlinear dynamics using only two phases and without additional radiation is required.

### RELATED ART DOCUMENT

### Patent Document

Korean Patent No. 10-2217392

### SUMMARY

An embodiment of the disclosure is to provide a deep learning-based multiphase coronary CT interpolation system and method, wherein it is possible to generate images (4D CCTA) of CCTA of intermediate phases from two different phases of coronary computed tomography angiography (CCTA) images in a cardiac cycle by utilizing deep learning.

According to an aspect of the disclosure, a deep learning-based multiphase coronary CT interpolation system is provided. The deep learning-based multiphase coronary CT interpolation system includes: an image processing unit that acquires two phases of coronary CT images as image data and performs preprocessing on the image data to generate preprocessed data; a deep learning inference unit that acquires a trained deep learning model and inputs the preprocessed data into the trained deep learning model to predict a 3D displacement map (registration field) at a specific time; and an output data generation unit that inputs the 3D displacement map to a spatial transformation layer (STL) to generate output data in the form of a CCTA image, wherein the output data includes a CCTA image for at least one arbitrary phase between the two phases of coronary CT images.

The two phases may refer to a pair of end-diastolic and end-systolic phases, and the image processing unit may include: an image data acquisition module that acquires a first phase image and a second phase image, which are the two phases of coronary CT images, as the image data; and an image data preprocessing module that performs preprocessing on the image data.

The image data preprocessing may include resizing or normalizing the image data.

the deep learning inference unit may include: a diffusion module that compresses/normalizes the preprocessed data to extract latent codes; a latent code interpolation module that acquires the latent codes and generates latent codes corresponding to an arbitrary time point; and a deformation module that compares the latent codes with a coronary artery CT image of the first of the two phases to predict the 3D displacement map.

The latent code interpolation module may acquire the two latent codes and the arbitrary time point t, a target time point, and perform interpolation to calculate a latent code at the time point t.

According to an aspect of the disclosure, a deep learning-based multiphase coronary CT interpolation method is provided. A deep learning-based multiphase coronary CT interpolation method includes: an image processing step of acquiring, by an image processing unit, two phases of coronary CT images as image data and performing preprocessing on the image data to generate preprocessed data; a deep learning inference step of acquiring, by a deep learning inference unit, a trained deep learning model and inputting the preprocessed data into the trained deep learning model to predict a 3D displacement map (registration field) at a specific time; and an output data generation step of inputting, by an output data generation unit, the 3D displacement map to a spatial transformation layer (STL) to generate output data in the form of a CCTA image, wherein the output data includes a CCTA image for at least one arbitrary phase between the two phases of coronary CT images.

A deep learning-based multiphase coronary CT interpolation system and method according to an embodiment of the disclosure can generate a full-phase (e.g., 10-phase) CCTA image from a two-phase CCTA image.

Furthermore, a deep learning-based multiphase coronary CT interpolation system and method according to an embodiment of the disclosure can reduce radiation exposure to the patient while simultaneously providing dynamic vascular image information, thereby providing information for more accurate diagnostic decisions.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of a deep learning-based multiphase coronary CT interpolation system according to an embodiment of the disclosure;

FIG. 2 is a block diagram of a image processing unit of FIG. 1;

FIG. 3 is a block diagram of a deep learning inference unit of FIG. 1;

FIG. 4 is a flowchart of a deep learning-based multiphase coronary CT interpolation method according to an embodiment of the disclosure;

FIG. 5 is a flowchart of step S11 of FIG. 4;

FIG. 6 is a flowchart of step S13 of FIG. 4;

FIG. 7 shows intermediate phase images automatically generated by interpolation between source and target images, as a result of a simulation of the disclosure; and

FIG. 8 shows quantitative indicators of the simulation results of FIG. 7.

### DETAILED DESCRIPTION

Hereinafter, some embodiments of the present disclosure will be described in detail with reference to exemplary drawings. When assigning reference numerals to components in each drawing, identical components may be assigned the same numerals, as much as possible, even if they are shown in different drawings. In addition, when describing the embodiments, detailed descriptions of related known structures or functions may be omitted if they are deemed to obscure the gist of the present technical concept. When terms such as “include,” “have,” and “composed of” are used herein, additional components may be added, unless “only” is used. When a component is expressed in the singular, it may also include plurals, unless otherwise explicitly stated.

Furthermore, terms such as “first,” “second,” “A,” “B,” “(a),” and “(b)” may be used to describe the components of the present disclosure. These terms are intended only to distinguish the components from other components and do not limit the nature, order, sequence, or number of the components.

In a description of the positional relationship between components, if two or more components are described as being “connected,” “coupled,” or “linked,” it may be understood that the two or more components may be directly “connected,” “coupled,” or “linked,” but should be understood that the two or more components may also be “connected,” “coupled,” or “linked” by being further “interposed” with another component. Here, another component may be included in one or more of the two or more components being “connected,” “coupled,” or “linked.”

In a description of the temporal relationship between components, their operating methods, or their manufacturing methods, for example, if the temporal or chronological relationship is described as “after,” “following,” “next to,” or “before,” then non-continuous cases may be included, as long as “immediately” or “directly” is not used.

Meanwhile, when numerical values or corresponding information (e.g., levels, etc.) for components are mentioned, even without separate explicit description, the numerical values or corresponding information may be interpreted as including an error range that may occur due to various factors (e.g., process factors, internal or external impact, noise, etc.).

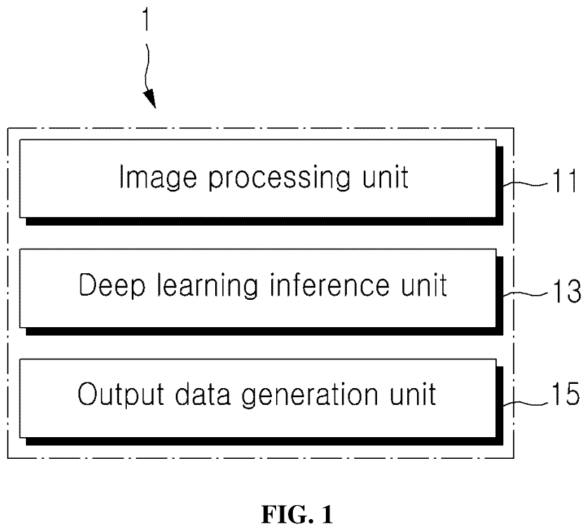

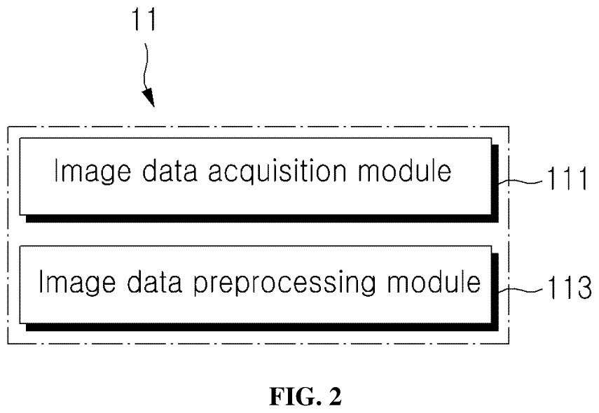

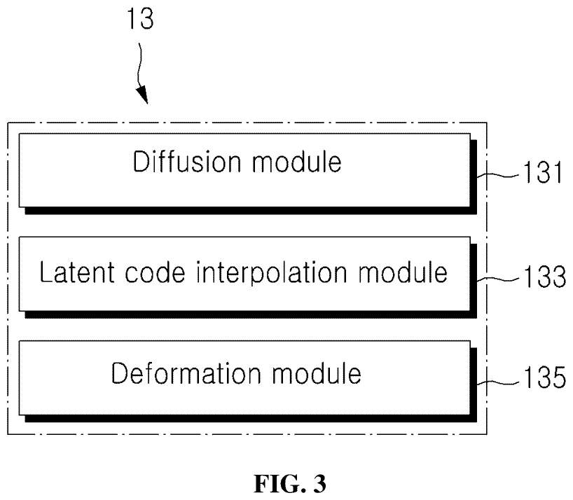

FIGS. 1 to 3 illustrate an embodiment of a deep learning-based multiphase coronary CT interpolation system of the disclosure. FIG. 1 is a block diagram of a deep learning-based multiphase coronary CT interpolation system according to an embodiment of the disclosure, FIG. 2 is a block diagram of an image processing unit of FIG. 1, and FIG. 3 is a block diagram of a deep learning inference unit of FIG. 1. Hereinafter, a deep learning-based multiphase coronary CT interpolation system of the disclosure will be described in detail using FIGS. 1 to 3.

A deep learning-based multiphase coronary CT interpolation system (1, hereinafter referred to as the “system” for convenience of explanation) according to an embodiment of the disclosure utilizes deep learning to generate, from two different phases of coronary CT (Coronary Computed Tomography Angiography, CCTA) images within the cardiac cycle, CCTA images of intervening phases. To this end, the system (1) according to an embodiment of the disclosure includes an image processing unit (11), a deep learning inference unit (13), and an output data generation unit (15), as illustrated in FIG. 1.

The image processing unit (11) acquires two phases of coronary CT images as image data and performs preprocessing on the image data to generate preprocessed data. More specifically, in an embodiment of the disclosure, the two phases may refer to end-diastolic and end-systolic phases. However, this is merely an embodiment of the disclosure, and the two phases may be arbitrarily set to different phases and used in the disclosure. To this end, the image processing unit (11) may include an image data acquisition module (111) and an image data preprocessing module (113), as illustrated in FIG. 2.

The image data acquisition module (111) may acquire two phases of coronary CT images as image data. Here, the CT images of respective phases acquired by the image data acquisition module (111) may be defined as a first phase image and a second phase image.

The image data preprocessing module (113) performs preprocessing on the image data. Here, the image data preprocessing module (113) may perform preprocessing on each of the first phase image and the second phase image. Specifically, in an embodiment of the disclosure, preprocessing of image data may mean resizing or normalizing the image data.

For example, in an embodiment of the disclosure, resizing the image data may involve standardizing the image data by adjusting it to a preset size, specifically, adjusting it to a size of 256×256×320.

Furthermore, in an embodiment of the disclosure, normalizing the image data may involve normalizing the pixel values contained in the image data to be values ranging from 0 to 1.

Once image data is acquired and preprocessed to generate preprocessed data, the deep learning inference unit (13) according to an embodiment of the disclosure acquires a trained deep learning model and inputs the preprocessed data into the trained deep learning model to predict a 3D displacement map (registration field) at a specific time. To this end, the deep learning inference unit (13) according to an embodiment of the disclosure includes a diffusion module (131), a latent code interpolation module (133), and a deformation module (135), as illustrated in FIG. 3. Furthermore, to acquire the trained deep learning model, the deep learning inference unit (13) according to an embodiment of the disclosure may further include a deep learning model training module (not shown).

The deep learning model training module acquires training data including preset labels and inputs the acquired training data into the deep learning model to train the corresponding deep learning model. Here, the training data, including the preset labels, may include the first and second phase images, as described above, and a t-phase image corresponding to an arbitrary time t between the two phases. Through this, the deep learning model training module of the disclosure may learn not only the first and second phase images, but also the t-phase image corresponding to an arbitrary time t, thereby generating a deep learning model enabling to derive the t-phase image as well as the first and second phase images.

While the deep learning inference unit (13) according to an embodiment of the disclosure has been described above as acquiring a deep learning model trained using the deep learning model training module, the disclosure is not limited thereto and may acquire a deep learning model trained externally.

The diffusion module (131) of the disclosure compresses/normalizes preprocessed data to extract latent codes. In an embodiment of the disclosure, the diffusion module (131) may compress the preprocessed first phase image and second phase image into low-dimensional latent codes using a 3D U-Net-based diffusion-type encoder. Through this, the diffusion module (131) of the disclosure can not only remove noise from an image, but also preserve anatomical features within the image. Furthermore, this operation may transform the volumes of two images into a latent representation appropriate for a unique coordinate system, thereby establishing a basis for interpolation.

The latent code interpolation module (133) acquires a latent code from the diffusion module (131) and uses it to generate a latent code corresponding to an arbitrary time point t. The latent interpolation module (133) according to an embodiment of the disclosure may acquire the two latent codes acquired from the diffusion module (131) and an arbitrary time point t, which is a target time point, and perform interpolation to calculate a latent code at the time point t.

More specifically, the latency interpolation module (133) of the disclosure calculates a latent code, which is the latent code of an intermediate phase, i.e., the time point t between two phases, and this is to first predict the intermediate time axis location in latency space to ensure structural consistency.

Finally, the deformation module (135) of the disclosure compares the latent code with the first phase image to predict a 3D displacement map. The deformation module (135) may channel-conjugate the latent code with the preprocessed first phase image and input it to a 3D CNN to predict a 3D displacement vector field that moves the preprocessed first phase image into the latent code. The deformation module (135) may use this 3D displacement vector field to generate a 3D displacement map, and this is to generate spatial deformation information for warping the first phase image, which is a source image, into an anatomical shape at the time t.

The output data generation unit (15) of the disclosure inputs a 3D displacement map into a spatial transformation layer (STL) to generate output data in the form of CCTA. Here, the output data may include a CCTA image for at least one arbitrary phase t between the first phase image and the second phase image.

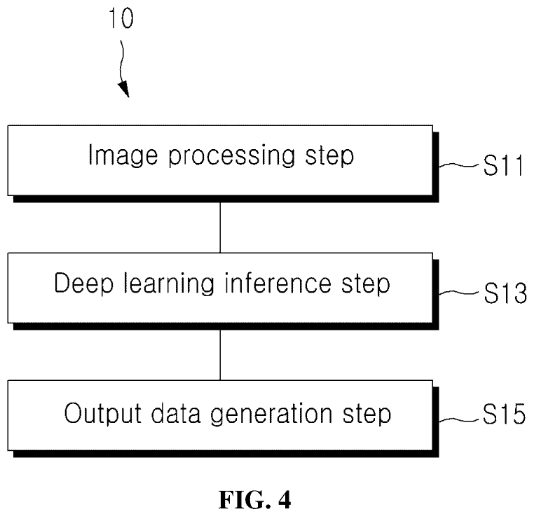

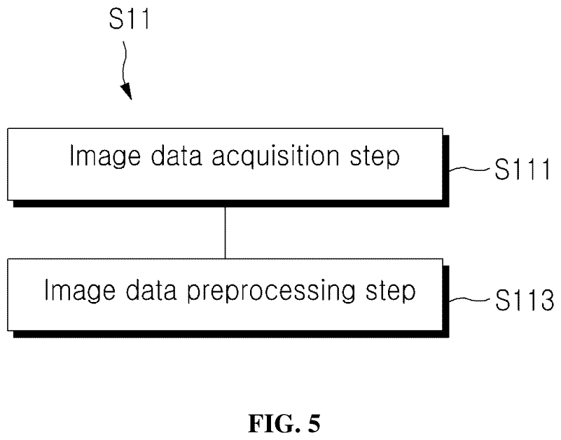

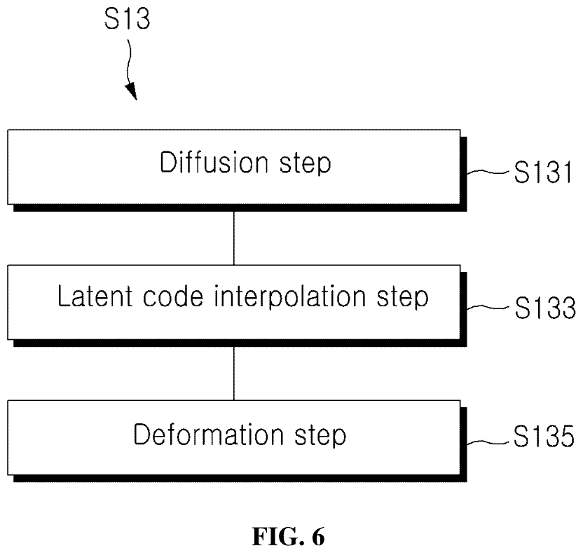

FIGS. 4 to 6 illustrate an embodiment of a deep learning-based multiphase coronary CT interpolation method of the disclosure. FIG. 4 is a flowchart of a deep learning-based multiphase coronary CT interpolation method according to an embodiment of the disclosure, FIG. 5 is a flowchart of step S11 of FIG. 4, and FIG. 6 is a flowchart of step S13 of FIG. 4. Hereinafter, a deep learning-based multiphase coronary CT interpolation method of the disclosure will be described in detail using FIGS. 5 and 6. Although FIG. 1 is used for convenience of explanation, the disclosure is not limited thereto, and various devices, systems, and terminals capable of performing similar functions or operations may be utilized.

A deep learning-based multiphase coronary CT interpolation method (10, hereinafter referred to as the “method” for convenience of explanation) according to an embodiment of the disclosure utilizes deep learning to generate, from two different phases of coronary CT (Coronary Computed Tomography Angiography, CCTA) images within the cardiac cycle, CCTA images of intervening phases. To this end, the method (10) according to an embodiment of the disclosure includes an image processing step (S11), a deep learning inference step (S13), and an output data generation step (S15), as illustrated in FIG. 4.

The image processing step (S11), the image processing unit (11) acquires two phases of coronary CT images as image data and performs preprocessing on the image data to generate preprocessed data. More specifically, in an embodiment of the disclosure, the two phases may refer to end-diastolic and end-systolic phases. However, this is merely an embodiment of the disclosure, and the two phases may be arbitrarily set to different phases and used in the disclosure. To this end, the image processing step (S11) may include an image data acquisition step (S111) and an image data preprocessing step (S113), as illustrated in FIG. 5.

The image data acquisition step (S111) may acquire two phases of coronary CT images as image data. Here, the CT images of respective phases acquired by the image data acquisition step (S111) may be defined as a first phase image and a second phase image.

The image data preprocessing step (S113) performs preprocessing on the image data. Here, the image data preprocessing step (S113) may perform preprocessing on each of the first phase image and the second phase image. Specifically, in an embodiment of the disclosure, preprocessing of image data may mean resizing or normalizing the image data.

For example, in an embodiment of the disclosure, resizing the image data may involve standardizing the image data by adjusting it to a preset size, specifically, adjusting it to a size of 256×256×320.

Furthermore, in an embodiment of the disclosure, normalizing the image data may involve normalizing the pixel values contained in the image data to be values ranging from 0 to 1.

Once image data is acquired and preprocessed to generate preprocessed data, the deep learning inference step (S13) according to an embodiment of the disclosure is configured such that a deep learning inference unit acquires a trained deep learning model and inputs the preprocessed data into the trained deep learning model to predict a 3D displacement map (registration field) at a specific time. To this end, the deep learning inference step (S13) according to an embodiment of the disclosure includes a diffusion step (S131), a latent code interpolation module (S133), and a deformation module (S135), as illustrated in FIG. 6. Furthermore, to acquire the trained deep learning model, the deep learning inference step (S13) according to an embodiment of the disclosure may further include a deep learning model training step (not shown).

The deep learning model training step acquires training data including preset labels and inputs the acquired training data into the deep learning model to train the corresponding deep learning model. Here, the training data, including the preset labels, may include the first and second phase images, as described above, and a t-phase image corresponding to an arbitrary time t between the two phases. Through this, the deep learning model training step of the disclosure may learn not only the first and second phase images, but also the t-phase image corresponding to an arbitrary time t, thereby generating a deep learning model enabling to derive the t-phase image as well as the first and second phase images.

While the deep learning inference step (S13) according to an embodiment of the disclosure has been described above as acquiring a deep learning model trained using the deep learning model training step, the disclosure is not limited thereto and may acquire a deep learning model trained externally.

The diffusion step (S131) of the disclosure compresses/normalizes preprocessed data to extract latent codes. In an embodiment of the disclosure, the diffusion step (S131) may compress the preprocessed first phase image and second phase image into low-dimensional latent codes using a 3D U-Net-based diffusion-type encoder. Through this, the diffusion step (131) of the disclosure can not only remove noise from an image, but also preserve anatomical features within the image. Furthermore, this operation may transform the volumes of two images into a latent representation appropriate for a unique coordinate system, thereby establishing a basis for interpolation.

The latent code interpolation step (133) acquires a latent code from the diffusion step (S131) and uses it to generate a latent code corresponding to an arbitrary time point t. The latent interpolation step (S133) according to an embodiment of the disclosure may acquire the two latent codes acquired from the diffusion step (S131) and an arbitrary time point t, which is a target time point, and perform interpolation to calculate a latent code at the time point t.

More specifically, the latency interpolation step (S133) of the disclosure calculates a latent code, which is the latent code of an intermediate phase, i.e., the time point t between two phases, and this is to first predict the intermediate time axis location in latency space to ensure structural consistency.

Finally, the deformation step (S135) of the disclosure is compares the latent code with the first phase image to predict a 3D displacement map. The deformation step (S135) may channel-conjugate the latent code with the preprocessed first phase image and input it to a 3D CNN to predict a 3D displacement vector field that moves the preprocessed first phase image into the latent code. The deformation step (S135) may use this 3D displacement vector field to generate a 3D displacement map, and this is to generate spatial deformation information for warping the first phase image, which is a source image, into an anatomical shape at the time t.

The output data generation step (S15) of the disclosure is configured such that the output data generation unit inputs a 3D displacement map into a spatial transformation layer (STL) to generate output data in the form of CCTA. Here, the output data may include a CCTA image for at least one arbitrary phase t between the first phase image and the second phase image.

In the simulation experiment of the disclosure, multiphase coronary CT data, reconstructed from a full cycle into 10 phases, was acquired from 10 patients, and eight sets were used for training, and two sets for validation. The diastolic phase images were used as the first phase image, and the systolic phase images were used as the second phase image, wherein the remaining eight phases of the 10 phases acquired for each set were used only as ground-truth intermediate phases.

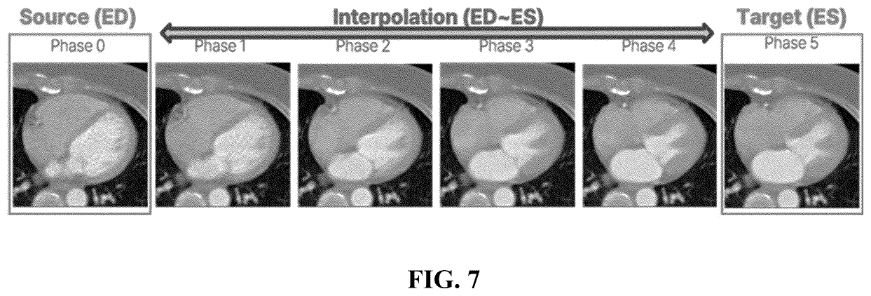

FIG. 7 illustrates these intermediate phase images automatically generated by interpolation between the source and target images. As shown in FIG. 7, when visually compared to the systolic phase, images of phases 1 to 4 demonstrate a gradual narrowing of the left ventricular lumen, a continuous shift of the coronary artery wall contour, and a seamless connection between the interventricular septum and myocardium. In other words, this simulation visually confirms that the 3D displacement map of the disclosure accurately reproduces nonlinear deformation while preserving anatomical structure, whereby it is possible to confirm that 3D CCTA images at all mid-cardiac cycle points can be reliably reconstructed using only two images which are diastolic and systolic.

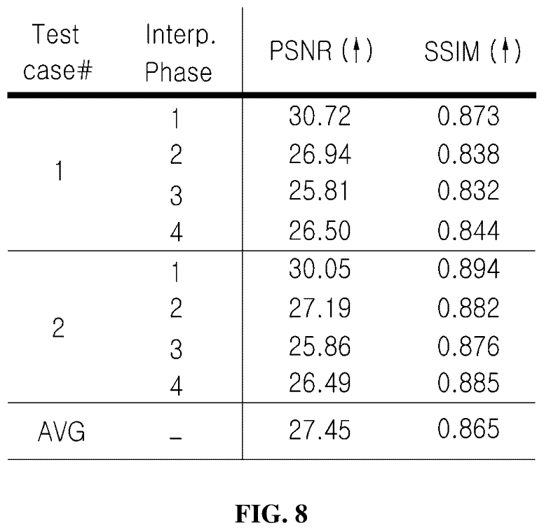

FIG. 8 illustrates the quantitative performance indicators of the simulation in FIG. 7, and specifically, shows the PSNR and SSIM values for each mid-phase of phases 1 to 4 with the overall average displayed in the AVG row.

The PSNR value stands for Peak Signal-to-Noise Ratio and represents the error in the reconstructed image relative to the original, calculated in dB, logarithmically. Here, a higher value indicates a closer reconstruction to the original. In medical imaging, a quality level above 25 dB is generally considered clinically useful.

The SSIM value is a value that compares the three elements of brightness/contrast/structure of an image between 0 and 1, and the closer it is to 1, the more identical the structure is. Generally, an SSIM of 0.8 or higher is used as a benchmark for assessing the preservation of structural information, such as coronary artery boundaries, myocardium, and serum differentiation.

Quantitative simulation results show an average PSNR of 27.45 dB, indicating excellent results even in 3D volumes with a wide HU range, such as CT. Furthermore, the average SSIM value is 0.865, indicating the anatomical continuity required for clinical interpretation.

That is, the simulation results provide quantitative evidence that the network quantitatively restored the mid-phase of the cardiac cycle to a level nearly identical to the original, even with input of only the first phase image and second phases image, which are images for two scenes of the diastole and systole.

Therefore, the system and method of the disclosure not only exhibit superior effectiveness compared to conventional ones, but also reduce the number of scans required, thereby providing dynamic vascular imaging information while reducing radiation exposure to the patient, and thus it is possible to provide information for more accurate diagnostic decisions.

The deep learning-based multiphase coronary CT interpolation method according to embodiments of the disclosure described above may be implemented as an application (computer program) stored on a storage medium of a computer.

Here, the computer may include a deep learning-based multiphase coronary CT interpolation system.

The computer's operating system may be a Windows or Macintosh operating system installed on a general PC, such as a desktop or laptop, or a mobile operating system, such as iOS or Android, installed on a mobile device, such as a smartphone or tablet PC.

The deep learning-based multiphase coronary CT interpolation method according to embodiments of the disclosure described above may be implemented as an application (i.e., a computer program) installed by default on a computer or by a user, and may be stored (recorded) on a computer-readable storage medium.

Likewise, in order for a computer to read a program recorded on a storage medium and execute the deep learning-based multiphase coronary CT interpolation method according to embodiments implemented as the program, the application (application program) described above may include codes coded in a computer language such as C, C++, JAVA, or machine language that can be read by a computer processor (CPU).

Such codes may include functional codes related to functions defining the aforementioned functions, and may also include control codes related to execution procedures necessary for the computer's processor to execute the aforementioned functions according to a predetermined procedure.

Furthermore, such codes may further include memory reference codes that specify the location (address) of the computer's internal or external memory where additional information or media required for the computer's processor to execute the aforementioned functions should be referenced.

Furthermore, if the computer's processor requires communication with another remote computer or server to execute the aforementioned functions, the codes may further include communication-related codes that specify how the computer's processor should communicate with another remote computer or server using the computer's communication module (e.g., wired and/or wireless communication module), and what information or media should be transmitted and received during the communication.

In addition, the functional program for implementing the present embodiments and codes and code segments related thereto may be easily inferred or changed by programmers in the technical field to which the disclosure belongs, taking into consideration the system environment of the computer that reads the storage medium and executes the program.

Additionally, a computer-readable storage medium recording the aforementioned program may be distributed across network-connected computer systems, allowing the computer-readable codes to be stored and executed in a distributed manner. In this case, one or more of the multiple distributed computers may execute some of the functions described above and transmit the results to one or more of the other distributed computers; furthermore, the computer receiving the results may also execute some of the functions described above and provide the results to the other distributed computers.

The computer-readable storage medium recording the application for executing the deep learning-based multiphase coronary CT interpolation method according to embodiments of the disclosure, as described above, may include, for example, ROM, RAM, CD-ROM, magnetic tape, floppy disk, or optical media storage devices.

Furthermore, a computer-readable storage medium recording an application program for executing the deep learning-based multiphase coronary CT interpolation method according to embodiments of the disclosure may be a storage medium (e.g., a hard disk) included in an application provider server, including an application store server, a web server related to the application or its service, or the like, or the application provider server itself, or may also be another computer or its storage medium recording the program.

A computer capable of reading a storage medium recording an application program for executing the deep learning-based multiphase coronary CT interpolation method according to embodiments of the disclosure may include not only general PCs such as desktops or laptops, but also mobile terminals such as smartphones, tablet PCs, Personal Digital Assistants (PDAs), and mobile communication terminals. Moreover, this should be interpreted as any computing-capable device.

The description above merely exemplifies the technical concepts of the disclosure, and those skilled in the art will appreciate that various modifications and variations can be made without departing from the essential characteristics of the disclosure. Therefore, embodiments disclosed herein are intended to illustrate, rather than limit, the technical concepts of the disclosure, and these embodiments do not limit the scope of the disclosure. The scope of protection of the disclosure should be construed in accordance with the following claims, and all technical concepts within the scope equivalent thereto should be construed as being included within the scope of the disclosure.

### EXPLANATION OF REFERENCE NUMERALS

1: deep learning-based multiphase coronary CT interpolation system
11: image processing unit
13: deep learning inference unit
15: output data generation unit
111: image data acquisition module
113: image data preprocessing module
131: diffusion module
133: latent code interpolation module
135: deformation module

What is claimed is:

1. A deep learning-based multiphase coronary CT interpolation system, comprising:
an image processing unit configured to acquire two phases of coronary CT images as image data and to perform preprocessing on the image data to generate preprocessed data;
a deep learning inference unit configured to acquire a trained deep learning model and to input the preprocessed data into the trained deep learning model to predict a 3D displacement map at a specific time; and
an output data generation unit configured to input the 3D displacement map to a spatial transformation layer (STL) to generate output data in a form of a CCTA image,
wherein the output data comprises the CCTA image for at least one arbitrary phase between the two phases of coronary CT images.

2. The deep learning-based multiphase coronary CT interpolation system of claim 1, wherein the two phases refer to a pair of end-diastolic and end-systolic phases, and the image processing unit comprises:
an image data acquisition module configured to acquire a first phase image and a second phase image, which are the two phases of coronary CT images, as the image data; and
an image data preprocessing module configured to perform the preprocessing on the image data.

3. The deep learning-based multiphase coronary CT interpolation system of claim 2, wherein the preprocessing on the image data comprises resizing or normalizing the image data.

4. The deep learning-based multiphase coronary CT interpolation system of claim 1, wherein the deep learning inference unit comprises:
a diffusion module configured to compress and normalize the preprocessed data to extract latent codes;
a latent code interpolation module configured to acquire the latent codes and to generate latent codes corresponding to an arbitrary time point; and
a deformation module configured to compare the latent codes with a coronary artery CT image of a first of the two phases to predict the 3D displacement map.

5. The deep learning-based multiphase coronary CT interpolation system of claim 4, wherein the latent code interpolation module is configured to acquire two latent codes and an arbitrary time point t as a target time point, and to perform interpolation to calculate a latent code at the arbitrary time point t.

6. A deep learning-based multiphase coronary CT interpolation method, comprising:
acquiring, by an image processing unit, two phases of coronary CT images as image data and performing preprocessing on the image data to generate preprocessed data;
acquiring, by a deep learning inference unit, a trained deep learning model and inputting the preprocessed data into the trained deep learning model to predict a 3D displacement map (registration field) at a specific time; and
inputting, by an output data generation unit, the 3D displacement map to a spatial transformation layer (STL) to generate output data in a form of a CCTA image,
wherein the output data comprises the CCTA image for at least one arbitrary phase between the two phases of coronary CT images.
